(19) United States
(12) Patent Application Publication
(10) Pub. No.: US 2026/0061838 A1
(43) Pub. Date: Mar. 5, 2026

(54) DRIVER BLOOD ALCOHOL CONTENT VERIFICATION FOR NON-ASSISTED, ASSISTED, AND AUTOMATED DRIVING SYSTEMS

(71) Applicant: GM GLOBAL TECHNOLOGY OPERATIONS LLC, Detroit, MI (US)

(72) Inventors: Daniel S. GLASER, Novi, MI (US); Joseph F. SZCZERBA, Grand Blanc, MI (US); Sean Taylor COUGHLIN, Shelby Township, MI (US); Sri Krishna Divya PEMMARAJU, Plymouth, MI (US); Pankti PUJARA, South Lyon, MI (US)

(21) Appl. No.: 18/817,880

(22) Filed: Aug. 28, 2024

(51) Int. Cl. B60K 28/06; A61B 5/00; A61B 5/1171; A61B 5/1172; B60W 60/00

(52) U.S. Cl. CPC B60K 28/06; A61B 5/1172; A61B 5/1176; A61B 5/4845; B60W 60/0051; A61B 2503/22

(57) ABSTRACT

A driver blood alcohol content (BAC) verification system for a host vehicle is disclosed. The driver BAC verification system includes: a device including a fingerprint sensor configured to detect a fingerprint of a first vehicle occupant, and a transdermal BAC sensor configured to detect a BAC level of the first vehicle occupant concurrently with the fingerprint sensor detecting the fingerprint; and a control module configured to, based on the fingerprint and the BAC level, verify that the BAC level is below a set threshold for being impaired, and in response to the BAC level being below the set threshold, permit the first vehicle occupant to drive the host vehicle.

### INTRODUCTION

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

The present disclosure relates to driver status monitoring and identification systems and assisted and automated driving systems.

Vehicles can have assisted and automated driving systems, which are implemented to aid a driver or vehicle occupant in driving a vehicle. As an example, a vehicle can have a hands-free driving system that controls steering, braking, and accelerating operations during at least a portion of a trip. During hands-free driving, a driver is not controlling driving operations but may intervene should the driver disagree with an action being performed by the vehicle. This may occur, for example, by the driver tapping on a brake pedal or gas pedal and taking over control of vehicle steering, braking, and accelerating operations.

### SUMMARY

A driver blood alcohol content (BAC) verification system for a host vehicle is disclosed. The driver BAC verification system includes: a device including a fingerprint sensor configured to detect a fingerprint of a first vehicle occupant, and a transdermal BAC sensor configured to detect a BAC level of the first vehicle occupant concurrently with the fingerprint sensor detecting the fingerprint; and a control module configured to, based on the fingerprint and the BAC level, verify that the BAC level is below a set threshold for being impaired, and in response to the BAC level being below the set threshold, permit the first vehicle occupant to drive the host vehicle.

In other features, the driver BAC verification system further includes a camera configured to detect an anatomical feature of the first vehicle occupant. The control module is configured to compare the detected anatomical feature and the detected fingerprint to a stored anatomical feature and fingerprint pair for the first vehicle occupant, and permit the first vehicle occupant to drive the host vehicle in response to verifying that the detected anatomical feature and detected fingerprint are for the first vehicle occupant.

In other features, the driver BAC verification system further includes a camera configured to detect a face of the first vehicle occupant in a driver seat of the vehicle. The control module is configured to compare the detected face and the detected fingerprint to a stored face and fingerprint pair for the first vehicle occupant, and permit the first vehicle occupant to drive the host vehicle in response to verifying that the detected face and detected fingerprint are for the first vehicle occupant.

In other features, the driver BAC verification system further includes a camera configured to detect an anatomical feature of a second vehicle occupant in a driver seat of the host vehicle. The control module is configured to compare the detected anatomical feature and the detected fingerprint to a stored anatomical feature and fingerprint pair for the second vehicle occupant, and, in response to determining that the detected fingerprint is not a fingerprint of the second vehicle occupant, at least one of i) limit driving operations, ii) maintain the host vehicle in a parked state, iii) disable driving operation of the host vehicle, and iv) transition to an autonomous drive mode.

In other features, the driver BAC verification system further includes a camera configured to detect a face of a second vehicle occupant in a driver seat of the host vehicle. The control module is configured to compare the detected face and the detected fingerprint to a stored face and fingerprint pair for the second vehicle occupant, and, in response to determining that the detected fingerprint is not a fingerprint of the second vehicle occupant, at least one of i) limit driving operations, ii) maintain the host vehicle in a parked state, iii) disable driving operation of the host vehicle, and iv) transition to an autonomous drive mode.

In other features, the control module is configured to detect that the first vehicle occupant is in a driver seat and is touching the fingerprint sensor without receiving feedback from a camera viewing the first vehicle occupant touching the fingerprint sensor.

In other features, the device is a start and stop switch of the host vehicle. The control module is configured, in response to the BAC level exceeding a the set threshold, at least one of i) prevent starting of the host vehicle, ii) maintain the host vehicle in parked state, and iii) limit operations of the host vehicle.

In other features, the control module is configured to setup the driver BAC verification system via a mobile application implemented in a module of the host vehicle or in a mobile device separate from the host vehicle.

In other features, the control module is configured to display the BAC level to the first vehicle occupant.

In other features, the control module is configured to: detect that a driver of the host vehicle has changed from the first vehicle occupant to a second vehicle occupant; detect a second fingerprint, a second BAC level and a second anatomical feature of the second vehicle occupant; and permit the second vehicle occupant to drive the host vehicle in response to verifying that the detected second fingerprint and the second anatomical feature are of the second vehicle occupant, and the second BAC level of the second vehicle occupant is below the set threshold.

In other features, a driver BAC verification method for a host vehicle is disclosed. The driver BAC verification method includes: detecting a fingerprint of a first vehicle occupant; detecting a BAC level of the first vehicle occupant concurrently with the detecting of the fingerprint; based on the fingerprint and the BAC level, verifying that the BAC level is below a set threshold for being impaired; and in response to the BAC level being below the set threshold, permitting the first vehicle occupant to drive the host vehicle.

In other features, the driver BAC verification method further includes: detecting an anatomical feature of the first vehicle occupant; comparing the detected anatomical feature and the detected fingerprint to a stored anatomical feature and fingerprint pair for the first vehicle occupant; and permitting the first vehicle occupant to drive the host vehicle in response to verifying that the detected anatomical feature and detected fingerprint are for the first vehicle occupant.

In other features, the driver BAC verification method further includes: detecting a face of the first vehicle occupant in a driver seat of the vehicle; comparing the detected face and the detected fingerprint to a stored face and fingerprint pair for the first vehicle occupant; and permitting the first vehicle occupant to drive the host vehicle in response to verifying that the detected face and detected fingerprint are for the first vehicle occupant.

In other features, the driver BAC verification method further includes: detect an anatomical feature of a second vehicle occupant in a driver seat of the host vehicle; comparing the detected anatomical feature and the detected fingerprint to a stored anatomical feature and fingerprint pair for the second vehicle occupant; and in response to determining that the detected fingerprint is not a fingerprint of the second vehicle occupant, at least one of i) limiting driving operations, ii) maintaining the host vehicle in a parked state, iii) disabling driving operation of the host vehicle, and iv) transitioning to an autonomous drive mode.

In other features, the driver BAC verification method further includes: detecting a face of a second vehicle occupant in a driver seat of the host vehicle; comparing the detected face and the detected fingerprint to a stored face and fingerprint pair for the second vehicle occupant; and in response to determining that the detected fingerprint is not a fingerprint of the second vehicle occupant, at least one of i) limiting driving operations, ii) maintaining the host vehicle in a parked state, iii) disabling driving operation of the host vehicle, and iv) transitioning to an autonomous drive mode.

In other features, the driver BAC verification method further includes detecting that the first vehicle occupant is in a driver seat and is touching a fingerprint sensor without receiving feedback from a camera viewing the first vehicle occupant touching the fingerprint sensor.

In other features, the driver BAC verification method further includes, in response to the BAC level exceeding the set threshold, at least one of i) preventing starting of the host vehicle, ii) maintaining the host vehicle in parked state, and iii) limiting operations of the host vehicle,

In other features, a start and stop switch of the host vehicle includes a fingerprint sensor for detecting the fingerprint and a transdermal sensor for detecting the BAC level.

In other features, the driver BAC verification method further includes setting up a driver BAC verification system, which is implementing the driver BAC verification method, via a mobile application implemented in a module of the host vehicle or in a mobile device separate from the host vehicle.

In other features, the driver BAC verification method further includes displaying the BAC level to the first vehicle occupant.

In other features, the driver BAC verification method further includes: detecting that a driver of the host vehicle has changed from the first vehicle occupant to a second vehicle occupant; detecting a second fingerprint, a second BAC level and a second anatomical feature of the second vehicle occupant; and permitting the second vehicle occupant to drive the host vehicle in response to verifying that the detected second fingerprint and the second anatomical feature are of the second vehicle occupant, and the second BAC level of the second vehicle occupant is below the set threshold.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 is a functional block diagram of a host vehicle including an example driver blood alcohol content (BAC) verification system with a BAC verification module in accordance with the present disclosure;

FIG. 2 is a functional block diagram of a portion of the driver BAC verification system of FIG. 1;

FIG. 3 is an example communication system including the host vehicle in accordance with the present disclosure; and

FIGS. 4A and 4B (collectively FIG. 4) illustrate a driver BAC verification method in accordance with the present disclosure.

In the drawings, reference numbers may be reused to identify similar and/or identical elements.

### DETAILED DESCRIPTION

Traditional vehicles permit access to and driving of a vehicle via a key fob. A user unlocks and enters the vehicle and presses a brake pedal and a start button to start the vehicle. A traditional vehicle is not equipped with sensors to detect a BAC level of the driver and/or a controller for limiting driving operations based on the detected BAC level of the driver. As a result, the vehicle may be driven while the driver is impaired and/or intoxicated (i.e., the driver has a BAC level that is greater than one or more BAC thresholds e.g., 0.2, 0.5, 0.8, 0.10, 0.15, which are associated with different levels of impairment.)

A hands-free driving system can control steering, braking, and accelerating of a host vehicle when engaged. A driver or vehicle occupant can disengage hands-free (sometimes referred to as assisted or automated) driving by, for example, tapping on a brake pedal or an accelerator pedal. Traditional hands-free driving systems have limited triggers for engaging and disengaging hands-free driving, limited or no interaction with a vehicle occupant, and limited functionality.

The examples set forth herein include a driver BAC verification system that is configured to detect a fingerprint, an anatomical feature (e.g., faceprint), and transdermal BAC of a user to detect whether the user is in a driver seat of a host vehicle and is impaired. The driver BAC verification system, based on this information, responds accordingly. For example, the driver BAC verification system may, in response to this information, determine whether to prevent a gear shifter from being shifted out of park, limit driving operations, perform autonomous driving operations, etc. The driver BAC verification system addresses the issues associated with determining whether a driver is attempting to put the vehicle into drive and is alcohol impaired.

To ensure that a transdermal BAC reading is associated with the person in the driver's seat of a host vehicle and not associated with another person in the vehicle, the fingerprint and transdermal BAC of the person is detected using a single combination fingerprint and transdermal sensing switch, which at the same time (or concurrently) detects the fingerprint and the BAC of the person. The fingerprint is then paired with an authenticated anatomical feature (e.g., an authenticated face) of the person, which is detected and recorded via one or more cameras of a driver monitoring system (DMS). The BAC verification system verifies whether the fingerprint and the anatomical feature are linked. Secure fingerprint and anatomical feature pairings are created and stored ahead of time in memory and used to determine if the detected fingerprint corresponds with the detected anatomical feature.

As an example, two individuals may purchase and/or have access to a same vehicle. The vehicle implements the BAC verification system disclosed herein. After purchasing the vehicle and/or after gaining initial access to the vehicle, each individual creates an anatomical feature (e.g., faceprint) and fingerprint pair via as described herein. One day, the first individual drinks heavily and is impaired and the second individual abstains from drinking alcoholic beverages. When getting into the vehicle, the first individual's anatomical feature is authenticated but as suspected does not pass the transdermal BAC test implemented using the transdermal BAC sensor. As an example, the fingerprint and transdermal BAC level of the first individual may be detected via sensors integrated in a switch (e.g., a start button switch or other switch).

As a result of not being able to start the vehicle, the first individual, without switching seats with the second individual, may ask the second individual to press the switch while being adamant that he or she should drive. To avoid conflict with the first individual, the second individual while not sitting in the driver seat presses the switch knowing that the BAC verification system will still detect the first individual in the driver seat and thus will not permit the first individual to drive the vehicle. The BAC verification system may display an indication that states the anatomical feature and fingerprint do not match and provides a message stating to make sure person in driver seat is touching the switch. The first individual then touches the switch, the anatomical feature and fingerprint match, and a BAC over-threshold or over-limit condition is detected. The BAC verification system may then lock a gear shifter in a parked state, transition a gear shifter to a parked state, and/or maintain a gear shifter in a parked state and/or perform one or more other limiting measures to prevent the driver from driving the vehicle and to prevent an accident from occurring. As a result, the first individual tells the second individual to drive and switches seats with the second individual. The second individual is able to be authenticated and pass the transdermal BAC test implemented by the BAC verification system. As a result, the vehicle starts and the second individual is able to safely drive the vehicle.

While the second individual is driving, the first individual may ask the second individual to pull-over and place vehicle in park but not turn off ignition. The first individual then switches seats with the second individual in another attempt to trick the system and drive. The switching of seats by the individuals may be detected via one or more cameras and/or seat sensors. The first individual is in the driver seat and the vehicle anatomical feature recognition system determines that the first individual is not the authenticated driver. As a result, the BAC verification system performs a limiting, counter and/or preventative measures, such as disabling the vehicle and/or performing one or more automated and/or autonomous driving operations (e.g., braking, accelerating, and/or steering). The first individual is again prevented from driving.

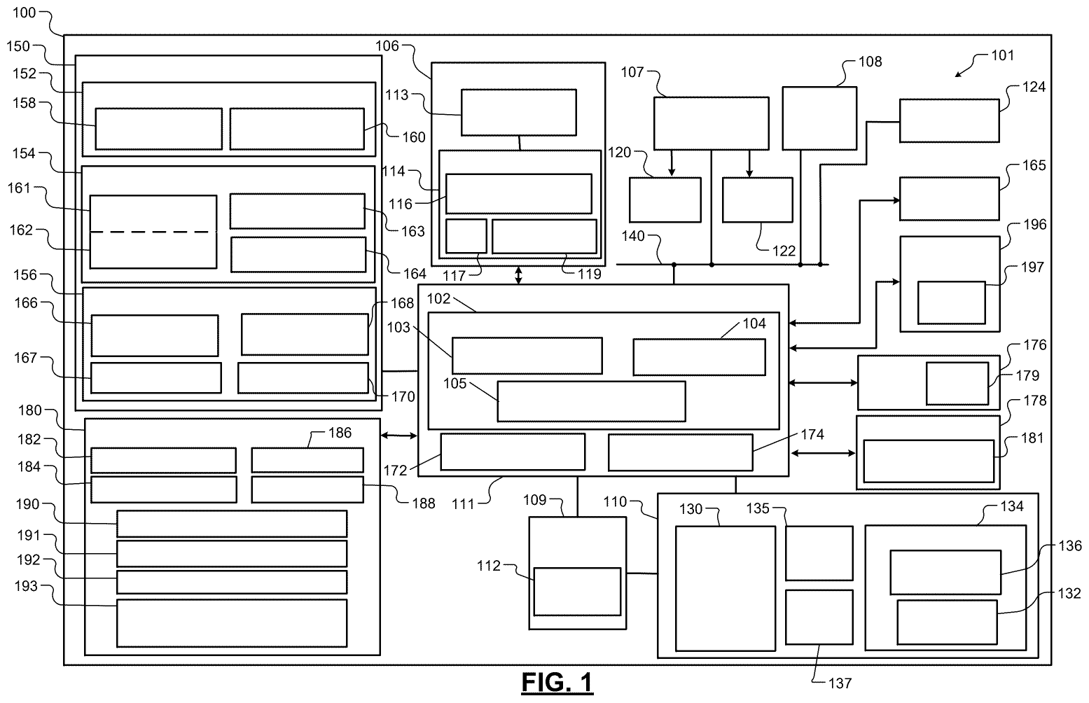

FIG. 1 shows a host vehicle 100 including an example driver BAC verification system 101 with a driving module 102. The driving module 102 includes a fingerprint module 103, a blood alcohol verification module 104, and an anatomical feature recognition module 105. Although shown as separate modules, two or more of the modules 102, 103, 104, 105 may be combined and implemented as a single module. The fingerprint module 103 detects, records, and verifies fingerprints. The BAC verification module 104 detects, records, and evaluates BAC levels. The anatomical feature recognition module 105 detects, records, and evaluates anatomical features.

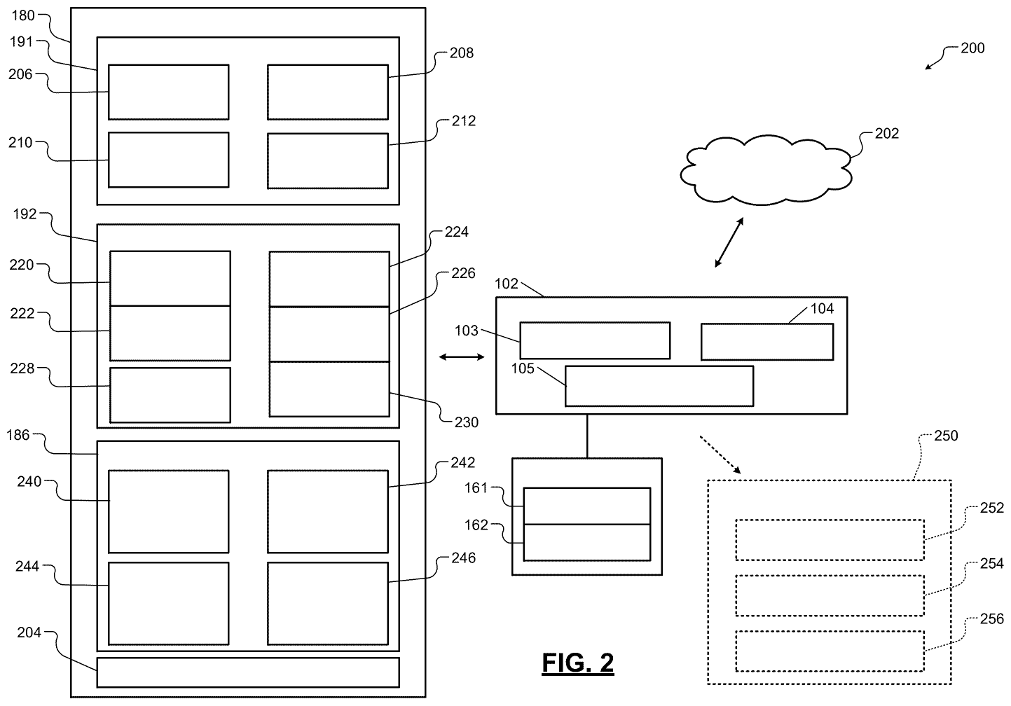

A portion of the driver BAC verification system 101 is shown in FIG. 1 and additional details of the driver BAC verification system 101 are shown in and/or described with respect to FIGS. 2-4. The host vehicle 100 includes a vehicle control module 111, which as shown includes the driving module 102. The driving module 102 performs: perception (or situation) determining operations; object detection, identification, classification, and graphical and visual identification operations; data look-up, collection, and gathering operations; interaction timing operations; assisted driving operations; image overlay operations; dialog operations including providing speech, text, and/or haptic messages; etc. The vehicle control module 111 may perform various operations based on the interaction with the user and the messages, generated as further described below.

The host vehicle 100 further includes one or more power sources 109, a telematics module 106, an infotainment module 107, other control modules 108 and a propulsion system 110. The vehicle control module 111 may control operation of the host vehicle 100 including the propulsion system 110 and other system described below. The power sources 109 may include one or more battery packs, a generator, a converter, a control circuit, terminals for high and low voltage loads, etc., as well as one or more battery sensors 112 for detecting states of the power sources 109 including voltages, current levels, states of charge, etc.

The telematics module 106 provides wireless communication services within the host vehicle 100 and wirelessly communicates with service providers, network devices, other vehicles, mobile devices, infrastructure devices, and other devices external and/or internal to the host vehicle 100. The telematics module 106 may support Wi-Fi®, Bluetooth®, Bluetooth Low Energy (BLE), Ultra Wideband (UWB), near-field communication (NFC), cellular, legacy (LG) transmission control protocol (TCP), long-term evolution (LTE), and/or other wireless communication and/or operate according to Wi-Fi®, Bluetooth®, BLE, UWB, NFC, cellular, and/or other wireless communication protocols. The telematics module 106 may include one or more transceivers 113 and a navigation module 114 with a global positioning system (GPS) and GNSS (or Global Navigation Satellite System) receiver 116. The navigation module 114 may include an inertial measurement unit (IMU) 117 and an odometer/wheel sensor 119. The transceivers 113 wirelessly communicate with network devices internal and external to the host vehicle 100 including cloud-based network devices, central stations, back offices, and portable network devices. The transceivers 113 may perform pattern recognition, channel addressing, channel access control, and filtering operations.

The navigation module 114 executes a navigation application to provide navigation services. The navigation services may include location identification services to identify where the host vehicle 100 is located. The navigation services may also include guiding a driver and/or directing the host vehicle 100 to a selected location. The navigation module 114 may communicate with a central station to collect map information indicating levels of traffic, transportation object identification and locations (e.g., locations and types of signs), path information, weather information, etc. As an example, if the host vehicle 100 is an assisted and/or automated driving vehicle, the navigation module 114 may direct the vehicle control module 111 along a selected route to a selected destination. The GPS and GNSS receiver 116 may provide vehicle velocity and/or direction (or heading) of the host vehicle 100 and other vehicles and objects (e.g., pedestrians and cyclists) and/or global clock timing information.

The infotainment module 107 may include and/or be connected to an audio system 122 and/or a video system including one or more displays (one display 120 is shown). The displays 120 and audio system 122 may be part of a human machine interface. The displays 120 may include cluster and/or center console displays, head-up displays, etc. Haptic devices 124 (e.g., steering wheel and/or seat vibration devices) may be used in addition to the displays and the audio system 122 to interact with a vehicle occupant such as a driver or passenger. This interaction is further described below. Messages may be displayed, audibly played out, and/or indicated via the displays 120, the audio system 122, the haptic devices 124, and/or via one or more other output devices.

The infotainment module 107 may provide various information, warnings, and proactive messages including: BAC content of a user; whether an anatomical feature of an individual in a driver seat matches a fingerprint of the individual in a driver seat; whether driving operations are limited and/or prevented; gear shifter status; upcoming and currently being performed operations (e.g., braking, accelerating, turning operations); detected objects (or obstacles); vehicle status information; diagnostic information; prognostic information; entertainment features and information; etc. The infotainment module 107 may be used to guide a vehicle operator to a certain location, indicate trip estimations (e.g., distances to selected destinations), and other information.

The propulsion system 110 may include one or more torque sources, such as one or more motors and/or one or more engines (e.g., internal combustion engines). In the example shown in FIG. 1, the host vehicle 100 includes an engine 130 and one or more motors 132. The torque sources are independently controlled. The propulsion system 110 includes a motor control system 134 that includes the one or more motors 132 and a motor control module 136 that may control operation of the one or more motors 132 based on signals from the vehicle control module 111. The propulsion system 110 may include a gear selector and/or shifter 135 for setting a gear of a transmission 137 and/or for setting a drive state of one or more of the torque sources. The gear selector and/or shifter 135 may be an electronic selector (e.g., one or more electrical switches), a mechanical and/or electrical shifter, or other selector and/or shifter.

The modules 102-108 and 111 may communicate with each other via one or more buses 140, such as a controller area network (CAN) bus and/or other suitable interface. The vehicle control module 111 may control operation of vehicle modules, devices and systems based on feedback from sensors 150.

The sensors 150 may include exterior sensors 152, interior sensors 154, and other sensors 156. The exterior sensors 152 may include radar and/or lidar sensors 158 and imaging and audio devices (e.g., visual spectrum cameras, long-wave infrared cameras, short-wave infrared cameras, ambient light sensors, and microphone or microphone array) 160. The exterior sensors 152 may be used to detect objects external to the host vehicle 100 and/or in a path of the host vehicle 100.

The interior sensors 154 may include a fingerprint sensor 161, a transdermal BAC sensor 162, one or more interior imaging sensors (e.g., cameras) 163, and a microphone or microphone array 164. The interior sensors 154 may be part of a driver monitoring system (DMS). In an embodiment, portions of the sensors 161, 162 overlap and/or are interlaid such that with a single touch by a finger tip on the surface including the sensors 161, 162, both a fingerprint and a BAC level can be detected for the individual that touched the surface.

The cameras 163 may be used to detect, track and/or monitor vehicle occupants including detecting locations of vehicle occupants in the vehicle, anatomical features (e.g., face, arms, hands, legs, etc.) of the vehicle occupants, head locations and/or eyes, etc. The cameras 163 may be located in various locations within the vehicle such as on a rear view mirror, on an A-pillar, on a dashboard, etc. As an example, facial features may be detected to identify a vehicle occupant. In an embodiment, a first camera is used for face recognition purposes. A second camera is used for detecting locations of vehicle occupant anatomy (e.g., head, arms, hands, legs, and feet). The second camera may be used to determine what each vehicle occupant is touching, such as a start/stop switch and/or other switch used for fingerprint and BAC determinations.

As an example, one or more switches 165 are included. In an embodiment, a single switch includes both fingerprint sensor and a transdermal BAC sensor. The single switch may be a start and stop switch or other type of switch. In another embodiment, a first switch includes both a fingerprint sensor a transdermal BAC sensor. By using a single switch, both a fingerprint and a BAC level of a user are able to be verified and evaluated and are automatically linked to the same person as only one person is able to touch the switch (or button of the switch) at a time. The one or more switches 165 may be located in various locations within the host vehicle 100 such as on a dashboard, a steering wheel, a center console, a door panel, an armrest, etc.

Location and movement of vehicle occupant head and eyes may be tracked. As an example, the interior sensors 154 may track posture, arm and hand locations, and eyes of a driver. This includes determining eye locations and eye gaze direction, detecting gestures made by the driver, detecting limb locations including finger locations, detecting orientation of a body of the driver, detecting speech of the driver, etc. This monitoring may be used to determine in which directions a vehicle occupant is looking, where the vehicle occupant is sitting, identification of the vehicle occupant, what object(s) the driver is looking at, etc.

The other sensors 156 may include a gear selector and/or shifter sensor 167, a vehicle speed sensor 166, acceleration sensors (e.g., longitudinal and lateral acceleration sensors) 168, and a fuel level sensor 170, as shown, and other sensors such as an inclinometer, an engine temperature sensor, and an engine oil pressure sensor. Additional sensors may also be included such as brake system sensors (a brake sensor 179 is shown) and steering system sensors (a steering angle sensor 181 is shown). The gear selector and/or shifter sensor 167 generates a signal indicative of a state of the gear selector and/or shifter 135.

The driving module 102 may use machine learning for facial recognition, determining locations of occupant limbs, for anatomical feature recognition, object classification including to identify and/or classify pedestrians, cyclists, and vehicles (e.g., oncoming traffic), as well as for probable trajectory determination of each detected, identified and/or classified object. The driving module 102 may determine the locations of objects based on feedback from the sensors 150.

The vehicle control module 111 may also include a mode selection module 172 and a parameter adjustment module 174. The mode selection module 172 may select a vehicle operating mode. The parameter adjustment module 174 may be used to adjust parameters of the host vehicle 100. The vehicle control module 111 may perform autonomous operations based on interaction with a vehicle occupant. As an example, the vehicle control module 111 may operate in a fully or partially autonomous mode and may control the propulsion system 110, a brake system 176, and a steering system 178. In an embodiment, the vehicle control module 111 controls operation of the systems 110, 176 and 178 based on or without interactions with a vehicle occupant. The vehicle control module 111 may i) perform autonomous operations such as steering, braking, accelerating, etc., and/or ii) display and/or audibly playout messages, perform haptic operations via haptic devices 124, and/or output messages and/or corresponding signals via other output devices.

In an embodiment, the driving module 102 uses computer vision, machine learning and cloud computing to identify, communicate, and evaluate scenarios where a moving host vehicle should yield to pedestrian(s), an obstructed roadway, and/or oncoming (right-of-way) traffic. The driving module 102 visualizes and takes into consideration in real-time pedestrians, roadway obstructions and oncoming traffic and performs operations to provide enhanced situation awareness to vehicle occupants.

The driving module 102 is configured to perceive the road ahead and surrounding areas based on outputs of sensors (e.g., cameras, radar sensors, and/or lidar sensors) and vehicle-to-everything (V2X) communication including vehicle-to-vehicle communication, vehicle-to-mobile device communication, vehicle-to-infrastructure communication, and other communication (e.g., vehicle to distributed network communication).

The host vehicle 100 may further include the memory 180. The memory 180 may store sensor data 182, parameters 184, applications 186, algorithms 188, historical data 190, on-board inputs 191, off-board inputs 192 from other devices external to the host vehicle 100 and other data 193. The data 182 may include fingerprint data, transdermal BAC data, occupant location data, anatomical feature data, etc. The parameters may include sensor parameters such as vehicle speed, vehicle acceleration, battery state of charge, fuel level, etc. applications 186. The applications 186 may include applications executed by the modules 102, 103, 104, 105, 107, 108, 111.

Although the memory 180 and the vehicle control module 111 are shown as separate devices, the memory 180 and the vehicle control module 111 may be implemented as a single device. The memory 180 may also store historical data 190 and other data 192 such as driver driving patterns, driver fueling patterns, driver stopping patterns, driver pickup patterns, other driver patterns, data collected by and/or generated by at least one of the modules 102, 111, traffic data, navigation data, map data, GPS data, path data, speed data, and acceleration data, etc.

The vehicle control module 111 may control operation of the propulsion system 110, the video system including the display 120, the audio system 122, the haptic devices 124, the brake system 176, the steering system 178, a seating system 196, and/or other devices and systems according to parameters set by the modules 102, 107, 108, 111, 174. The seating system 196 may include seat sensors 197 for detecting presence of an occupant and/or change in occupants, for example, change in a driver. The vehicle control module 111 may set at least some of the parameters based on signals received from the sensors 150.

The vehicle control module 111 may receive power from the power sources 109, which may be provided to the propulsion system 110, the brake system 176, the steering system 178, the seating system 196, etc. Power supplied to the haptic devices 124, the motors 132, the brake system 176, the steering system 178, the seating system 196, and/or actuators thereof may be controlled by the vehicle control module 111 to, for example, adjust: motor speed, torque, and/or acceleration; braking pressure; steering wheel angle; pedal position; state of haptic devices 124; etc. This control may be based on the outputs of the sensors 150, the navigation module 114, the GPS and GNSS receiver 116, the data and information received from external devices, and the data and information stored in the memory 180.

The vehicle control module 111 may determine various parameters including a vehicle speed, a motor speed, a gear state, an accelerator position, a brake pedal position, an amount of regenerative (charge) power, an amount of auto start/stop discharge power, and/or other information. The vehicle control module 111 may control operations of the systems 110, 176, 178 based on the stated parameters. The driving module 102 may display vehicle status information based on the stated parameters.

The driving module 102 monitors real-time behavior of vehicle occupants including locations, anatomical features, limb locations, speech, gaze patterns, head positions, gestures (e.g., hand gestures, finger gestures, facial gestures, etc.). Gaze patterns include directions of an occupant's head and eyes. This information is used to determine where, how, duration, timing, and intensity of alerts and other conversational messages.

The host vehicle 100 can include various systems for assisting a driver, for performing autonomous operations, and/or for indicating to a vehicle occupant information regarding an environment of the host vehicle. For example, a host system may include a navigation system that provides map information indicating lane boundaries, street locations, speed limits, geographical locations of selected destinations, etc. The host system may provide the driver with instructions for driving to a selected destination and/or may perform autonomous operations such as braking, steering, and accelerating operations to drive the vehicle to the destination based on the map information.

As another example, the host vehicle 100 may include object detection and collision warning systems for detecting impending objects and performing countermeasures and/or taking evasive action to prevent a collision. The vehicle control module 111 determines locations of the objects relative to the host vehicle 100 and trajectories of the objects and the host vehicle 100. If it is determined that the host vehicle 100 is likely to collide with one of the objects, one or more warning signals may be generated to indicate to the driver and/or the object of concern of the potential collision. These warnings may be provided in addition to digital gateways and other information described herein. The vehicle control module 111 may also or alternatively perform one or more other countermeasures (e.g., apply brakes to decelerate the host vehicle, change a steering angle of the host vehicle, etc.) to prevent a collision.

FIG. 2 shows a portion 200 of the driver BAC verification system 101 of FIG. 1. The portion 200 includes the driving module 102, the memory 180, and a distributed communications system 202. The driving module 102 includes the modules 103, 104, and 105. The memory 180 stores the on-board inputs 191, the off-board inputs 192, the algorithms 186 and user profiles 204.

The on-board inputs 191 include transdermal BAC sensor inputs 206, fingerprint sensor inputs 208, camera inputs 210, and switch inputs 212 (e.g., state of a start and stop switch). The switch inputs 212, when having integrated fingerprint and transdermal BAC sensors, may include fingerprint and transdermal BAC sensor inputs. As an example, a switch 215 is shown including the fingerprint sensor 161 and the transdermal BAC sensor 162. The switch 215 may be a start and stop switch or other switch.

The off-board inputs 192 include GPS data 220, traffic data 222, V2X data 224, a road database 226, weather information 228, and WiFi data 230. The algorithms 186 include: a fingerprint algorithm 240 for identifying an individual based on a detected fingerprint; an anatomical feature recognition algorithm 242 for recognizing anatomical features associated with an individual; a transdermal BAC algorithm 244 for determining a BAC of an individual based on a galvanic skin response of the individual; and a drive monitoring algorithm 246 for monitoring driving behavior of driver. The galvanic skin response has an associated chemical signature that can be read via the transdermal BAC sensor 162.

The user profiles 204 include fingerprints, anatomical feature information, etc. of users. In an embodiment, each user profile 204 includes a fingerprint and anatomical feature pair, such as a fingerprint and facial feature. The anatomical feature information may include: facial feature sizes, shapes, colors, etc.; image pixel colors; image pixel patterns; limb sizes, shapes, colors, etc.; and/or other anatomical feature information. The driving module 102 links fingerprints and anatomical features of individuals and stores these pairings as a part of user profiles 204 in memory 180. The driving module 102 may communicate with devices external to the host vehicle via the distributed communications system 202. This includes sharing and receiving data and information, some of which is referred to herein. Based on the inputs 191, 192 and user profiles 204, the driving module 102 performs various system operations 250, some of which are shown in FIG. 2 including gear selector interlock setting 252, vehicle driving disabling and/or limiting 254, and automated (or autonomous) driving 256.

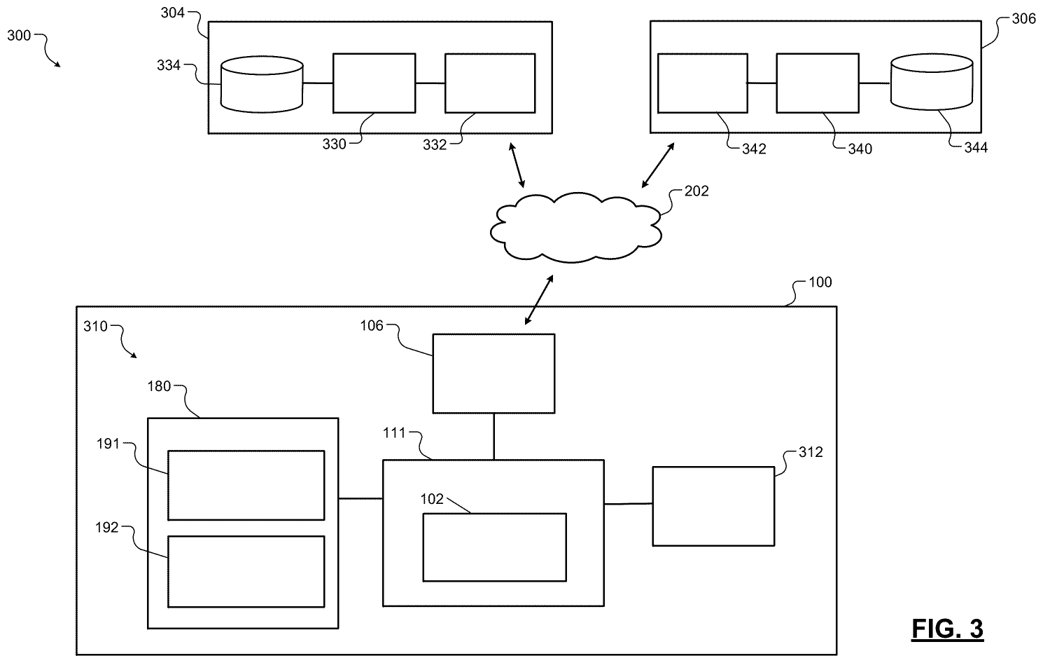

FIG. 3 shows a communication system 300 including the host vehicle 100, the distributed communications system 202, a cloud-based network device 304, and a back office 306. A portion 310 of the driver BAC verification system 101 of FIG. 1 is shown. The portion 310 includes the vehicle control module 111, the telematics module 106, the memory 180, and an HMI 312, which may include any interfacing devices referred to herein including displays, speakers, haptic devices, lights, etc. The driving module 102 may utilize a windshield as a display and provide an augmented reality via an augmented reality heads up display (ARHUD). The vehicle control module 111 includes the driving module 102.

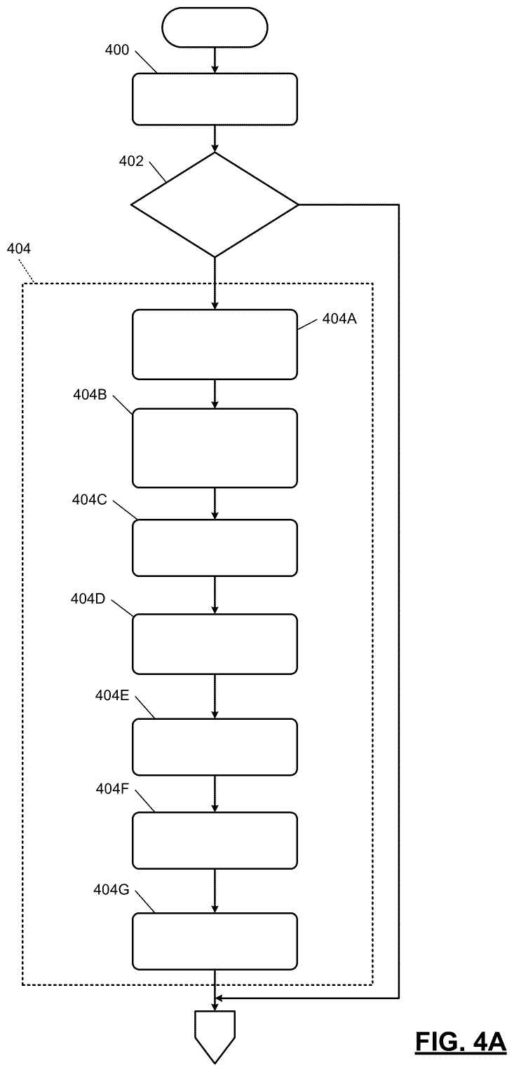

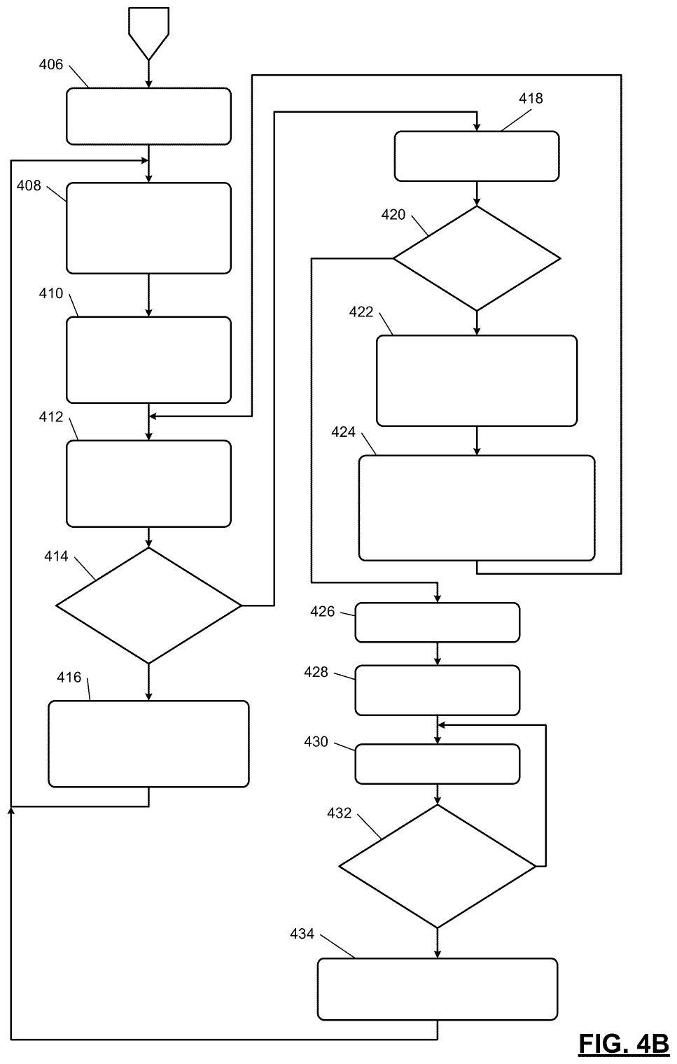

The memory 180 includes on-board inputs 191 and the off-board inputs 191. The on-board inputs 191 may include fingerprint data, BAC data, anatomical data, posture, arm, hand and head positions, eye positions, and gaze angle of a vehicle occupant, automated driving system status information, vehicle braking information, steering angle information, object detection information, vehicle acceleration information, etc. This information may be provided via the sensors referred to herein. The off-board inputs 191 may include: GPS information; information received via the Internet; and/or information received via V2X communication, WiFi communication, cellular communication, and/or satellite communication. The off-board inputs 191 may include information received from the cloud-based network device 204 and/or the back office 306. In an embodiment, the driving module 102 may perform a system setup via a mobile application implemented in the vehicle and/or on a mobile device separate from and in communication with the vehicle. This may include downloading to the driving module 102 and/or memory 180 BAC set thresholds, actions to perform when the BAC set thresholds are reached, anatomical feature and fingerprint pairs of users, etc. An example system setup is described below with respect to operation 404 of FIG. 4.

The cloud-based network device 304 may include a control module 330, a transceiver 332, and a database 334. One or more cloud-based network devices may be included and include one or more edge devices. The back office 306 may include a control module 340, a transceiver 342, and a database 344.

The devices 304, 306 may create, store, and modify occupant preference profiles, each of which being specific to a particular person (driver and/or vehicle occupant). A profile may include preferences with regards to alert messages, when and what type of alert message to provide, whether the person likes reactive messages, whether the person likes proactive messages, etc. As an example, a profile may indicate if a person has loss of hearing, limited feeling in legs or rear, etc. such that certain types of alerts should not be provided. A profile may indicate if a driver typically looks at a particular display and provide alerts on that display. A profile may be accessed for a driver based on the driver's dedicated identifier (ID), using face recognition, fingerprint recognition, voice recognition, a username, a password, etc. The profiles are shared with the host vehicle 100 and may be created, stored and/or modified by the vehicle, such as by the driving module 102 and/or one of the modules 103, 104, 105 of FIG. 1. The preferences may be used and updated during each drive cycle. The preferences may also be shared with multiple vehicles. A driver may switch vehicles and the driver's preferences may be provided to and used by each of the vehicles.

The driving module 102 may utilize computer vision, machine learning, V2X communication, and cloud computing to highlight objects within a scenario which the host vehicle 100 should yield to including pedestrian(s), obstructing roadway objects, oncoming (right-of-way) traffic, and/or other objects. Machine learning algorithms may be used to classify types and trajectories of objects located within range of the planned pathway of host vehicle 100. The driving module 102 may provide a graphical user-interface to convey presence and intention (i.e., predicted paths) of detected objects. The driving module 102 may track locations of the host vehicle 100 and other objects, speed of host vehicle 100 and other objects, trajectories of the host vehicle 100 and other objects, roadway curvatures, vehicle steering, etc. and display images via the HMI 312 based on these locations, speeds, trajectories, and roadway curvatures. The driving module 102 may also display the images having selected type, size, shape, and color based on the stated information and occupant position, host vehicle location, and other vehicle sensed aspects of the driving environment.

FIG. 4 shows a driver BAC verification method. The following operations may be iteratively performed. The operations may be performed by the modules 102-105 of FIGS. 1-3.

At 400, the driving module 102 detects a user entering the host vehicle 100. At 402, the driving module 102 determines whether to perform a system setup process. The system setup process may include creating an anatomical feature and fingerprint pairing for a particular user. The system setup process may be performed initially when it is a first time that the user has entered the vehicle, when it is a first time that the user is to drive the vehicle, when the vehicle is purchased, etc. If yes, operation 404 is performed, otherwise operation 406 is performed.

At 404, the driving module 102 performs the system setup process. At 404A, the driving module 102 instructs the user to follow instructions to make an anatomical feature and fingerprint pairing. The instructions may be provided visually on a display and/or audibly. As an example, the driving module 102 may instruct the user to press a button, such as a start and stop switch, which includes a fingerprint sensor. This may occur prior to performing operation 404D. Other instructions may also be provided.

At 404B, the anatomical feature recognition module 105 detects anatomical features of the user via one or more cameras. This may include detecting any of the anatomical features referred to herein.

At 404C, the anatomical feature recognition module 105 records the anatomical feature data in memory. At 404D, the fingerprint module 103 detects a fingerprint of the user via a fingerprint sensor in the switch pushed by the user.

At 404E, the fingerprint module 103 records the fingerprint in the memory. At 404F, the driving module 102 links the anatomical feature information to the fingerprint to create an anatomical feature and fingerprint pairing, which are stored as part of a profile of the user in memory at 404G.

At 406, the driving module 102 identifies the user based on the anatomical feature recognition. This may include recognizing an anatomical feature of the user based on camera data and user profile data associated with the anatomical feature.

At 408, the driving module 102 detects pressing of button of a switch (e.g., one of the switches 165 of FIG. 1) and collects outputs of the sensors of the switch. The switch may include fingerprint sensor and transdermal BAC sensor. One or more switches may have been pressed. In one embodiment, a start and stop button is pressed. In another embodiment, a fingerprint sensing and transdermal BAC sensing pad is touched. The pad may not be a switch. The transdermal BAC sensor may be referred to as a transdermal BAC reader. In another embodiment, the first switch is implemented as a start and stop switch and another switch includes the fingerprint and transdermal BAC sensors.

At 410, the modules 103, 104 record the fingerprint and transdermal BAC level detected at 408. At 412, the driving module 102 determines whether anatomical feature and fingerprint are linked in a stored user profile for the same person.

At 414, the driving module 102 determines if the user for which a fingerprint was provided at 408 is the person currently in the driver seat. If not, operation 416 is performed, otherwise operation 418 is performed.

At 416, the driving module 102 displays a request that the user in driver seat press button, switch, pad, or other device that has both the fingerprint sensor and transdermal BAC sensor.

At 418, the driving module 102 may then display the driver's BAC level. At 420, the driving module 102 may determine if the BAC level is above a set threshold associated with being impaired. If yes, operation 422 is performed, otherwise operation 426 is performed.

At 422, the driving module 102 maintains a gearshift interlock in a locked state, limits driving operations, and/or autonomously drives the vehicle to a safe location. The limited driving operations may include limiting vehicle speed, traveled distance, etc. When operating in a limited driving (or limp) mode, the vehicle may be able to move at a slow speed to a safe location.

At 424, the driving module 102 may display the gear selector (or shifter) status, the limited driving operations available, whether autonomous driving is enabled and/or active, a request to change drivers, and/or other related information.

At 426, the driving module 102 may unlock the gear shift interlock. At 428, the driving module 102 may permit driving of the vehicle by the user in the driver seat. At 430, the driving module 102 may monitor the driver and driving behavior.

At 432, the driving module 102 may determine whether the person in the driver seat (referred to as the previous driver) has changed to another person (referred to as the current and/or new driver). If yes, operation 434 may be performed, otherwise operation 430 may be performed.

At 434, the driving module 102 may i) limit vehicle driving operations and/or disable driving operations, and/or ii) implement automated and/or autonomous driving. This may occur until the new driver's BAC level is checked and verified to below a set threshold for impairment. Operation 408 may be performed after operation 434 to detect the BAC of the new driver.

The above-described operations are meant to be illustrative examples. The operations may be performed sequentially, synchronously, simultaneously, continuously, during overlapping time periods or in a different order depending upon the application. Also, any of the operations may not be performed or skipped depending on the implementation and/or sequence of events.

The fingerprint sensor (or scanner) and transdermal BAC sensor (or reader) referred to herein may be integrated and/or embedded into a surface of a vehicle start button or a surface of any other vehicle control device. Fingerprint and faceprint pairs may be created and stored as part of user profiles. The fingerprint and faceprint pairs enable real time fingerprint and faceprint authentication.

When a face recognition camera and/or other camera is used for detecting other anatomical features, machine learning may be implemented to identify each vehicle occupant and to distinguish between the occupants in the vehicle. Machine learning may be implemented to determine, for example, what device each occupant is touching. Human body models may be stored and used to distinguish anatomical features, body parts and limbs of different occupants.

In an embodiment, a face of a person in a driver seat is present and authenticated before the driving module 102 permits the person to drive the vehicle. In an embodiment, the face of the person must be present and authenticated when a start and stop button is pressed and the vehicle is shifted out of park.

In an embodiment, when the driver of the vehicle changes to another person, the face of the driver changes and the vehicle is operated in a limp mode limiting driving. Other or additional limiting and/or preventative measures may be taken such as generating warnings, disabling the vehicle, and/or performing other limiting and/or preventative measures. The vehicle is maintained in this state until the BAC of the new driver is verified to be associated with non-impairment. In an embodiment, when the face changes due to a change in drivers while driving, the driving module 102 places the vehicle in an automated driving mode for autonomous driving.

In an embodiment, the driving module 102 is configured to identify the person in the driver seat based on outputs of the fingerprint sensor without a camera that is positioned to view the person and the surface where the fingerprint sensor is located at the same time. A camera is used, for example, to recognize a face of the person in the driver seat and then the fingerprint and the faceprint are compared to linked pairs to verify if the fingerprint belongs to the person in the driver seat. In another embodiment, a camera is included that can view the person in the seat touching the surface of the fingerprint sensor.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A driver blood alcohol content (BAC) verification system for a host vehicle, the driver BAC verification system comprising:
a device comprising
a fingerprint sensor configured to detect a fingerprint of a first vehicle occupant, and
a transdermal BAC sensor configured to detect a BAC level of the first vehicle occupant concurrently with the fingerprint sensor detecting the fingerprint; and
a control module configured to, based on the fingerprint and the BAC level, verify that the BAC level is below a set threshold for being impaired, and in response to the BAC level being below the set threshold, permit the first vehicle occupant to drive the host vehicle.

2. The driver BAC verification system of claim 1, further comprising a camera configured to detect an anatomical feature of the first vehicle occupant,
wherein the control module is configured to compare the detected anatomical feature and the detected fingerprint to a stored anatomical feature and fingerprint pair for the first vehicle occupant, and permit the first vehicle occupant to drive the host vehicle in response to verifying that the detected anatomical feature and detected fingerprint are for the first vehicle occupant.

3. The driver BAC verification system of claim 1, further comprising a camera configured to detect a face of the first vehicle occupant in a driver seat of the vehicle,
wherein the control module is configured to compare the detected face and the detected fingerprint to a stored face and fingerprint pair for the first vehicle occupant, and permit the first vehicle occupant to drive the host vehicle in response to verifying that the detected face and detected fingerprint are for the first vehicle occupant.

4. The driver BAC verification system of claim 1, further comprising a camera configured to detect an anatomical feature of a second vehicle occupant in a driver seat of the host vehicle,
wherein the control module is configured to compare the detected anatomical feature and the detected fingerprint to a stored anatomical feature and fingerprint pair for the second vehicle occupant, and, in response to determining that the detected fingerprint is not a fingerprint of the second vehicle occupant, at least one of i) limit driving operations, ii) maintain the host vehicle in a parked state, iii) disable driving operation of the host vehicle, and iv) transition to an autonomous drive mode.

5. The driver BAC verification system of claim 1, further comprising a camera configured to detect a face of a second vehicle occupant in a driver seat of the host vehicle,
wherein the control module is configured to compare the detected face and the detected fingerprint to a stored face and fingerprint pair for the second vehicle occupant, and, in response to determining that the detected fingerprint is not a fingerprint of the second vehicle occupant, at least one of i) limit driving operations, ii) maintain the host vehicle in a parked state, iii) disable driving operation of the host vehicle, and iv) transition to an autonomous drive mode.

6. The driver BAC verification system of claim 1, wherein the control module is configured to detect that the first vehicle occupant is in a driver seat and is touching the fingerprint sensor without receiving feedback from a camera viewing the first vehicle occupant touching the fingerprint sensor.

7. The driver BAC verification system of claim 1, wherein:
the device is a start and stop switch of the host vehicle; and
the control module is configured, in response to the BAC level exceeding a the set threshold, at least one of i) prevent starting of the host vehicle, ii) maintain the host vehicle in parked state, and iii) limit operations of the host vehicle.

8. The driver BAC verification system of claim 1, wherein the control module is configured to setup the driver BAC verification system via a mobile application implemented in a module of the host vehicle or in a mobile device separate from the host vehicle.

9. The driver BAC verification system of claim 1, wherein the control module is configured to display the BAC level to the first vehicle occupant.

10. The driver BAC verification system of claim 1, wherein the control module is configured to:
detect that a driver of the host vehicle has changed from the first vehicle occupant to a second vehicle occupant;
detect a second fingerprint, a second BAC level and a second anatomical feature of the second vehicle occupant; and
permit the second vehicle occupant to drive the host vehicle in response to verifying that the detected second fingerprint and the second anatomical feature are of the second vehicle occupant, and the second BAC level of the second vehicle occupant is below the set threshold.

11. A driver blood alcohol content (BAC) verification method for a host vehicle, the driver BAC verification method comprising:
detecting a fingerprint of a first vehicle occupant;
detecting a BAC level of the first vehicle occupant concurrently with the detecting of the fingerprint;
based on the fingerprint and the BAC level, verifying that the BAC level is below a set threshold for being impaired; and
in response to the BAC level being below the set threshold, permitting the first vehicle occupant to drive the host vehicle.

12. The driver BAC verification method of claim 11, further comprising:
detecting an anatomical feature of the first vehicle occupant;
comparing the detected anatomical feature and the detected fingerprint to a stored anatomical feature and fingerprint pair for the first vehicle occupant; and
permitting the first vehicle occupant to drive the host vehicle in response to verifying that the detected anatomical feature and detected fingerprint are for the first vehicle occupant.

13. The driver BAC verification method of claim 11, further comprising:
detecting a face of the first vehicle occupant in a driver seat of the vehicle;
comparing the detected face and the detected fingerprint to a stored face and fingerprint pair for the first vehicle occupant; and
permitting the first vehicle occupant to drive the host vehicle in response to verifying that the detected face and detected fingerprint are for the first vehicle occupant.

14. The driver BAC verification method of claim 11, further comprising:
detect an anatomical feature of a second vehicle occupant in a driver seat of the host vehicle;
comparing the detected anatomical feature and the detected fingerprint to a stored anatomical feature and fingerprint pair for the second vehicle occupant; and
in response to determining that the detected fingerprint is not a fingerprint of the second vehicle occupant, at least one of i) limiting driving operations, ii) maintaining the host vehicle in a parked state, iii) disabling driving operation of the host vehicle, and iv) transitioning to an autonomous drive mode.

15. The driver BAC verification method of claim 11, further comprising:
detecting a face of a second vehicle occupant in a driver seat of the host vehicle;
comparing the detected face and the detected fingerprint to a stored face and fingerprint pair for the second vehicle occupant; and
in response to determining that the detected fingerprint is not a fingerprint of the second vehicle occupant, at least one of i) limiting driving operations, ii) maintaining the host vehicle in a parked state, iii) disabling driving operation of the host vehicle, and iv) transitioning to an autonomous drive mode.

16. The driver BAC verification method of claim 11, further comprising detecting that the first vehicle occupant is in a driver seat and is touching a fingerprint sensor without receiving feedback from a camera viewing the first vehicle occupant touching the fingerprint sensor.

17. The driver BAC verification method of claim 11, further comprising, in response to the BAC level exceeding the set threshold, at least one of i) preventing starting of the host vehicle, ii) maintaining the host vehicle in parked state, and iii) limiting operations of the host vehicle,
wherein a start and stop switch of the host vehicle comprises a fingerprint sensor for detecting the fingerprint and a transdermal sensor for detecting the BAC level.

18. The driver BAC verification method of claim 11, further comprising setting up a driver BAC verification system, which is implementing the driver BAC verification method, via a mobile application implemented in a module of the host vehicle or in a mobile device separate from the host vehicle.

19. The driver BAC verification method of claim 11, further comprising displaying the BAC level to the first vehicle occupant.

20. The driver BAC verification method of claim 11, further comprising:
detecting that a driver of the host vehicle has changed from the first vehicle occupant to a second vehicle occupant;
detecting a second fingerprint, a second BAC level and a second anatomical feature of the second vehicle occupant; and
permitting the second vehicle occupant to drive the host vehicle in response to verifying that the detected second fingerprint and the second anatomical feature are of the second vehicle occupant, and the second BAC level of the second vehicle occupant is below the set threshold.
